(19) United States
(12) Patent Application Publication
(10) Pub. No.: US 2010/0261980 A1
(43) Pub. Date: Oct. 14, 2010

(54) METHOD AND APPARATUS FOR DETECTING AN ABNORMAL SITUATION

(75) Inventors: Yang Peng, Shanghai (CN); Sheng Jin, Shanghai (CN); Warner Rudolph Theophile ten Kate, Eindhoven (NL); Heribert Baldus, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/678,499

(22) PCT Filed: Sep. 8, 2008
(86) PCT No.: PCT/IB2008/053614

§ 371 Date: Jun. 18, 2010

(30) Foreign Application Priority Data
Sep. 19, 2007 (CN) 200710153386.X

(51) Int. Cl. A61B 5/00

(52) U.S. Cl. 600/301

(57) ABSTRACT

To improve the power efficiency of a monitoring system, especially for worn devices, the present invention provides a monitoring system (300) comprising a physiological signal monitor (310) configured to monitor at least one physiological signal; a processor (320) configured to receive the output signal of the physiological signal monitor and detect an abnormal occurrence of at least one physiological signal; and a movement detection sub-system (330) coupled to receive the output signal of the processor and configured to monitor the movement of a target body, based on the output signal of the processor, for detecting the abnormal situation. The power consumption of the whole system can be decreased by using the monitoring result of physiological signals as a trigger for the movement detection sub-system.

### FIELD OF THE INVENTION

The present invention generally relates to methods and apparatus for detecting an abnormal situation, more particularly falls, in a human being.

### BACKGROUND OF THE INVENTION

Healthcare is becoming increasingly important, especially for seniors and patients. Among all the potential risks, fall, which is defined as a sudden, uncontrolled and unintentional downward displacement of the body to the ground, causes injuries to millions of people every year. Fall is the most important reason for losing independence and one of the top-three causes of death among seniors.

Different detection solutions are already available. Most of them can be categorized as worn devices and environment-based detection systems. Environment-based solutions usually have camera and/or vibration sensors installed in people's homes and do not require too many power-saving schemes. Worn-device systems, which usually comprise accelerometers and tilt sensors, are much more sensitive to power consumption. In general, a worn-device system can be used for several months without changing the battery or recharging. There is a need to extend the lifetime of a worn-device system without reducing the speed and accuracy of detecting a possible fall.

U.S. patent application US20030153836A1 discloses a method of improving the accuracy of detecting a possible fall, by introducing monitoring physiological information after an abnormal movement has been detected by an actimetric sensor. FIG. 1 shows its method. The analysis of the actimetric information 12 may be of three types: normal 111, in which only the actimetric sensors function; evidently abnormal 112, in which one passes directly to stage 13 for generating an alarm; and potentially abnormal 113, in which a significant movement has been detected without being certain whether it involves a fall. In this situation 113, a supplementary stage 14 is implemented for confirmation or invalidation of the abnormality of the situation. The physiological information 15 is taken into account to confirm or invalidate the abnormality. In the case of invalidation, it returns to the normal situation 111. In the opposite case, it passes to generate an alarm automatically or manually.

However, the method of U.S.20030153836 cannot satisfy the needs of reducing power consumption. Thus there is a need to find a power-efficient solution without decreasing the detection accuracy.

### SUMMARY OF THE INVENTION

One aspect of some embodiments of the present invention provides a power-efficient and detection-accurate method and apparatus for detecting an abnormal situation, falls in particular, in a human being.

In accordance with some embodiments of the invention, a monitoring system for monitoring an abnormal situation of a target body is provided, the monitoring system comprising: a physiological signal monitor configured to monitor a physiological signal; a processor configured to receive the output signal of the physiological signal monitor and detect an abnormal occurrence of the physiological signal; and a movement detection sub-system coupled to receive the output signal of the processor and work in a selected detection mode for monitoring the movement of the target body, based on the output signal of the processor, for detecting the abnormal situation.

In normal cases, the movement detection sub-system can work in a low power-consumption and low sampling mode. If an abnormality of one or more physiological signals is detected after their analysis, the movement detection sub-system can be instructed to work at a higher sampling rate mode so as to accurately detect the abnormality, particularly the physical movement, of the patient. Both power consumption and detection accuracy are thus taken into consideration.

Optionally, the physiological signal monitor comprises one or more biosensors, each detecting one physiological signal. The physiological signal may be any one of heart beat, blood pulse, blood pressure, ECG, EMG, SPO₂ (sphygmous oxygen saturation), or any other signal representing the target body's physiological activity.

Optionally, the processor comprises a detector configured to detect the abnormal occurrence of the physiological signal on the basis of the output signal of the physiological signal monitor, and a mode selector configured to generate a mode selection signal for causing the movement detection sub-system to operate in a corresponding detection mode. It is advantageous to adapt the working mode of the movement detection sub-system to the status of the physiological signals, so that the power consumption can be saved considerably, especially when there is no abnormal situation.

Based on the detection result, the detection mode can be selected from, but not limited to, at least one of off, sleep, doze, normal and active modes. Each mode is characterized by the sampling rate or power consumption level.

Optionally, the monitoring system may further comprise one or more environment sensors configured to monitor the environment in which the target body is located. The output signal or signals of the environment sensor or sensors can be sent to the processor so as to detect a change of environment. The system thus provides the advantages of taking such a change of environment into consideration when selecting the detection mode of the movement detection sub-system.

Optionally, the monitoring system may further comprise a transmitter which is configured to store and transmit the detection results of the movement detection sub-system and/or the physiological signal monitor. Analysis of the detection result of the physiological signal can be used to instruct the transmitter to operate in a store mode or in a transmission mode.

In accordance with some embodiments of the present invention, a monitoring method comprises the steps of: a) monitoring a physiological signal; b) detecting an abnormal occurrence of the physiological signal; and c) monitoring physical movement of a target body in a detection mode corresponding to the output signal of step b).

Optionally, the monitoring method may further comprise the step of monitoring a change of environment and the step of selecting a detection mode, while taking both the abnormal occurrence of the physiological signal and the change of environment into consideration.

The present invention is based on the recognition that the detection result, especially detection of the occurrence of an abnormality of a physiological signal or signals, is used to set the detection mode of the movement detection sub-system. When the physiological signal or signals are normal, the movement detection sub-system can operate at a lower sampling rate and a lower power consumption. When the physiological signal varies within a wide range, e.g. when the patient is exercising, the movement detection sub-system operates at a higher sampling rate, and the power consumption consequently rises. In the case of an abnormality of the physiological signal, e.g. a sudden rise of blood pressure and/or heart beat, the movement detection sub-system operates at a much higher sampling rate and is sensitive to the patient's physical movement.

Other objects and effects of the present invention will become apparent from the following description and the appended claims when taken in conjunction with the accompanying drawings.

### BRIEF DESCRIPTION OF THE DRAWINGS

FIG. 1 illustrates the method disclosed in US20030153836A1;

FIG. 2 illustrates an embodiment of the present invention of setting an accelerometer's working mode based on the output of monitoring an ECG sensor;

FIG. 3 illustrates a monitoring system in accordance with one embodiment of the present invention;

FIG. 4 illustrates a monitoring method in accordance with one embodiment of the present invention.

Throughout the above Figures, the same or similar reference numerals will be understood to refer to the same or similar features or functions.

### DESCRIPTION OF EMBODIMENTS

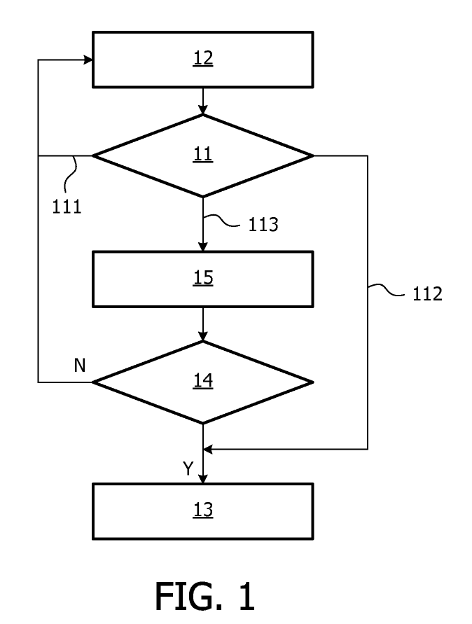

In the embodiment of FIG. 1, the physiological signal is monitored to validate whether a real fall occurs so as to improve the accuracy of fall detection. In the whole process, the actimetry works in a full mode, i.e. there is no power saving.

The invention is based on the recognition that one or more physiological signals are monitored to detect a possible abnormal situation, especially a fall. When at least one physiological signal detects an abnormality, the movement detection sub-system is set into different working modes so as to accurately detect the abnormal situation. In view of the factors that cause falls, physiological signals can be continuously measured for certain patients, e.g. those suffering from chronic diseases like hypertension. Instead of the methods of continuously monitoring the body movement and orientation, the apparatus and methods disclosed in the present invention can continuously measure the necessary physiological signals of a user and thus make an initial assessment of the likelihood of falling. For example, dizziness raises the risk of falling; blood pressure may help to detect such a phenomenon; a large deviation of the normal pulse oximetry or heart beat may indicate a higher risk; a sustained increase of EMG (electromyogram) activity may imply a risk of falling. In the case of abnormal physiological signals, indicating an increased risk of an abnormal situation, the movement detection sub-system will further switch to different modes.

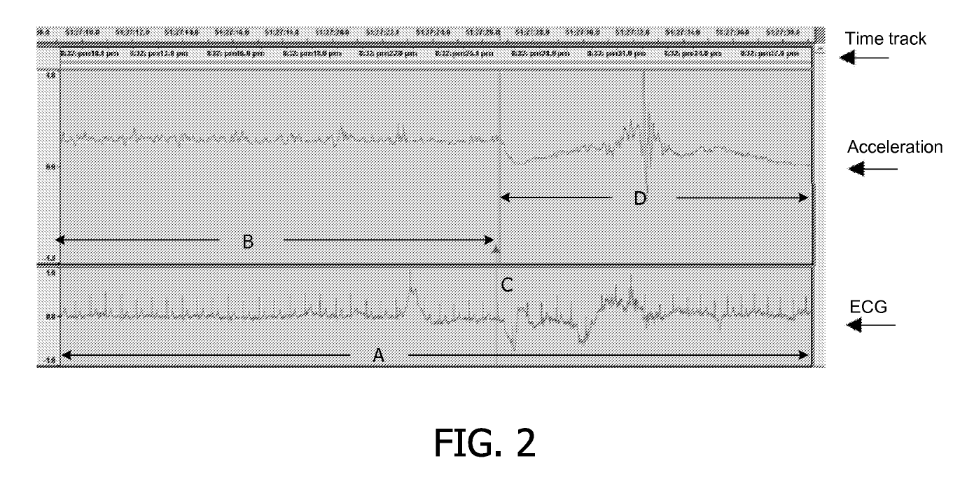

An embodiment is illustrated in FIG. 2 for better understanding of the invention. The movement detection sub-system, e.g. the accelerometer or meters and tilt sensor or sensors, can be operated in the following modes.

Off mode: the accelerometer and tilt sensor are turned off and not working;

Sleep mode: only one accelerometer is working, at a low sampling rate, e.g. 5 Hz, and the processor of the movement detection sub-system is also working at a lower speed;

Doze mode: the accelerometer and the tilt sensor are working at a higher sampling rate, e.g. 20 Hz;

Normal mode: the accelerometer and the tilt sensor are working at a normal sampling rate, e.g. 50 Hz, and the processor of the movement detection sub-system is working at a power-saving speed, e.g. at half the highest speed;

Active mode: the accelerometer and the tilt sensor are working at the highest sampling rate, e.g. 100 Hz, and the processor of the movement detection sub-system is also working at the highest speed in order to detect a fall quickly.

An ECG (electrocardiogram) signal is taken as an example in this embodiment. In the normal case, the ECG sensor works in the full mode to detect the ECG signal of a patient, as shown in the bottom of this Figure and labeled as A. When there is no abnormality, the accelerometer works in the doze mode at a sampling rate of 20 Hz, as shown in the left part of the Figure and labeled as B. When an abnormality of the ECG signal is detected, shown in the middle of the Figure and labeled as C, the accelerometer switches to the active mode at a sampling rate of 100 Hz, shown in the right part of the Figure and labeled as D. It is easy to understand from this embodiment that, in the normal case, the power consumption of the monitoring system can be decreased considerably. When an abnormality occurs, the monitoring system can quickly switch to a more accurate monitoring mode without losing its detection accuracy.

In other cases, when a person is sleeping, his physiological signals indicate less movement, which implies less risk of falling. The movement detection sub-system can then be switched to a less accurate mode. When the person is moving, e.g. walking or running, which implies a greater risk of falling; the movement detection sub-system can be switched to a more accurate mode.

Besides physiological signals, environment factors can also be used to indicate the possibility of a fall occurring. In a corresponding manner, one or more environment sensors can be used to monitor the environment continuously or discontinuously. For example, a light sensor can be used to detect whether the environment is too dark. If it is too dark, the movement detection sub-system can be switched to a more precise working mode. A temperature sensor can also play a similar role. In another embodiment, the working modes of the environment sensors can be set in dependence upon the output of monitoring the physiological signals. For example, if it is detected that the patient is asleep, the light sensor can be set to work in the off mode; if it is detected that the patient is walking very fast or running, the light sensor can also be set to work in the off mode or the doze mode, because people normally walk fast or run in a light rather than in a dark environment.

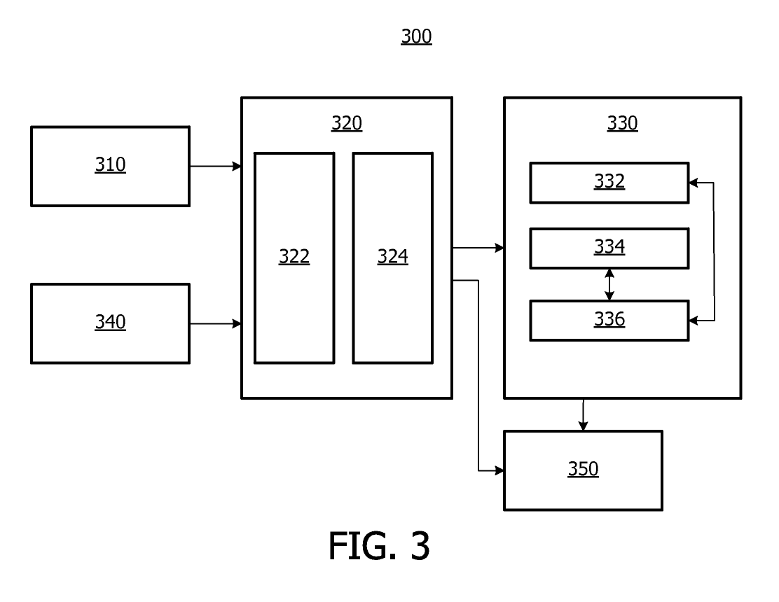

FIG. 3 illustrates a monitoring system in accordance with one embodiment of the present invention. The monitoring system 300 comprises a physiological signal monitor 310, a processor 320 and a movement detection sub-system 330. The physiological signal monitor 310 can be used to monitor one or more physiological signals, each physiological signal representing one physiological character of the target body. For example, the physiological signal may be any one of heart beat, blood pulse, blood pressure, ECG, EMG, SPO₂, or any other signal representing the target body's physiological activity. The processor 320 can be used to receive the output signal of the physiological signal monitor 310 and detect an abnormal occurrence of one or more physiological signals. The movement detection sub-system 330 is coupled to receive the output signal of the processor 320 and monitor the movement of the target body, based on the output signal of the processor, for detecting the abnormal situation.

By using the monitoring system 300, it is advantageous to use the monitoring result of the physiological signal monitor 310 as a trigger for setting the working mode of the movement detection sub-system 330 and thus save power of the whole system. When these physiological signals show no abnormality, which normally means that the target body is in a good condition, the movement detection sub-system 330 can work at a lower sampling rate, i.e. a power-saving mode.

In another embodiment, the processor 320 may further comprise a detector 322 and a mode selector 324. The detector 322 is configured to detect the abnormal occurrence of one or more physiological signals on the basis of the output signal of the physiological signal monitor 310. The mode selector 324 is configured to generate a mode selection signal for causing the movement detection sub-system 330 to operate in a corresponding working mode. It is also practical to configure the processor 320 to forward the output signal of the physiological signal monitor 310 to the movement detection sub-system 330, which may be further used to help improve the detection accuracy.

In another embodiment, the movement detection sub-system 330 may further comprise one or more accelerometers 332, one or more tilt sensors 334 and a second processor 336. Each accelerometer 332 can be used to measure the acceleration of the target body. Each tilt sensor 334 can be used to measure the tilting level of the target body. The second processor 336 can be used to process the output signal of the accelerometer or meters and the tilt sensor or sensors so as to detect the abnormal situation. The accelerometer 332, the tilt sensor 334 and the second processor 336 can be used as the currently available devices. Furthermore, the second processor 336 can be configured to detect the abnormal situation while taking the output signal of the physiological signal monitor 310 into consideration.

The movement detection sub-system 330 can be configured to operate in different working modes. Each working mode is characterized by its sampling rate, power consumption, or both. For example, the movement detection sub-system 330 can work in any one of off, sleep, doze, normal and active modes.

In another embodiment, one or more environment sensors 340 can be incorporated in the monitoring system 300 for utilizing the change of environment so as to improve the detection accuracy and power consumption efficiency. The output signal of the environment sensor 340 is coupled to the processor 320 so as to detect the change of environment. It is also practical to forward the output signal of the environment sensor 340 to the movement detection sub-system 330 through the processor 320.

In another embodiment, the monitoring system may further comprise a transmitter 350 which can be configured to store and/or transmit the output signal of the movement detection sub-system. If the output signals of the physiological signal monitor 310 and/or the environment sensor 340 are forwarded to the movement detection sub-system 330, it is practical for the transmitter 350 to store and/or transmit the output signals of the physiological signal monitor 310 and/or the environment sensor 340. It is advantageous to control the working mode of the transmitter 350 on the basis of the output of the processor and on the abnormal occurrence of the physiological signals and/or a change of environment. If there is no abnormality in the physiological signals and no considerable change of environment, the transmitter 350 works in the store mode, i.e. it only saves the output signal of movement detection sub-system 330 and/or the output signals of physiological signal monitor 310 and environment sensor 340. If there is an abnormality or a considerable change of environment, the transmitter 350 switches to the transmission mode so as to transmit the detected signal in real time, for example, to a doctor or any other rescue center. It is advantageous to notify the real-time detection result and get help for the patient.

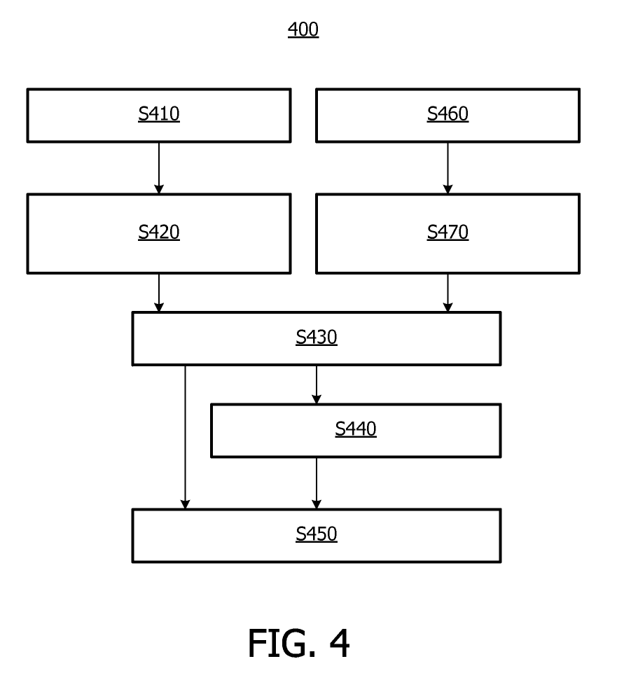

FIG. 4 illustrates a method of monitoring an abnormal situation in accordance with one embodiment of the present invention. In the method 400, the physiological signal or signals is/are monitored in step S410 so as to obtain the current physiological activity of the target body. In step S420, it is detected whether there is an abnormal occurrence of one or more physiological signals. If an abnormal occurrence is detected, in step S430, the detection mode of a movement detection device/system is selected. In step S440, the movement detection device/system thus works in the selected detection mode. In step S450, the output signal obtained in step S440 can be stored or transmitted. Also, transmission of the signal obtained in step S450 can be controlled on the basis of the output in step S430. It is further practical to incorporate the detection of the environment. In step S460, the environment, in which the target body is located, is monitored. In step S470, it is detected whether there is a considerable change of environment. The output signal obtained in step S470 can be incorporated into step S430 so as to help select the detection mode, which further helps to improve the detection accuracy.

By using the systems and methods proposed by the present invention, it is advantageous to use the abnormal occurrence of physiological signals for triggering the movement detection sub-system, which normally consumes more power. The power of the whole system thus decreases. It is also advantageous to combine the monitored physiological signals with the detection result of the movement detection so as to improve the detection accuracy. It is also advantageous to take the change of environment into account so that more energy can be saved and the movement detection can be improved in due time.

The above embodiments have been described by way of illustrative examples only and are not intended to limit the technical approach of the present invention. It will be evident to those skilled in the art that the technical approach of the present invention can be modified without departing from the spirit and scope of the present invention and the appendent claims.

1. A monitoring system for monitoring an abnormal situation of a target body, the monitoring system comprising: a physiological signal monitor configured to monitor a physiological signal; a processor configured to receive the output signal of the physiological signal monitor and detect an abnormal occurrence of the physiological signal; and a movement detection sub-system coupled to receive the output signal of the processor and work in a selected detection mode for monitoring the movement of the target body, based on the output signal of the processor, for detecting the abnormal situation.

2. The monitoring system according to claim 1, wherein the physiological signal monitor comprises a biosensor configured to detect the physiological signal.

3. The monitoring system according to claim 1, wherein the physiological signal is any one of heart beat, blood pulse, blood pressure, ECG, EMG, SPO₂, or any signal representing the target body's physiological activity.

4. The monitoring system according to claim 1, wherein the processor comprises: a detector configured to detect the abnormal occurrence of the physiological signal on the basis of the output signal of the physiological signal monitor; and a mode selector configured to generate a mode selection signal for causing the movement detection sub-system to operate in a corresponding detection mode.

5. The monitoring system according to claim 4, wherein the processor is further configured to forward the output signal of the physiological signal monitor to the movement detection subsystem.

6. The monitoring system according to claim 1, wherein the movement detection subsystem is configured to work in a plurality of detection modes, each detection mode being characterized by at least any one of the sampling rate and the power consumption level.

7. The monitoring system according to claim 6, wherein each detection mode is any one of off, sleep, doze, normal and active modes.

8. The monitoring system according to claim 6, wherein the movement detection sub-system comprises: at least one accelerometer configured to measure an acceleration of the target body; at least one tilt sensor configured to measure a tilting level of the target body; and a second processor configured to process the output signals of the accelerometer or meters and the tilt sensor or sensors to detect the abnormal situation.

9. The monitoring system according to claim 1, wherein the abnormal situation is a fall of the target body.

10. The monitoring system according to claim 9, further comprising at least one environment sensor configured to monitor the environment in which the target body is located, wherein the processor is further configured to detect a change of environment on the basis of the output signal of the environment sensor and generate a mode selection signal on the basis of the detection result of the change of environment and an abnormal occurrence of at least one physiological signal.

11. The monitoring system according to claim 10, wherein the environment sensor is configured to monitor at least any one of light, temperature, and humidity.

12. The monitoring system according to claim 1, further comprising a transmitter configured to store and transmit at least any one of the output signals of the physiological signal monitor and the movement detection sub-system, wherein the transmitter is further configured to operate in a store mode or in a transmission mode on the basis of the output signal of the processor.

13. A method of monitoring an abnormal situation of a target body, the method comprising the steps of: a) monitoring a physiological signal; b) detecting an abnormal occurrence of the physiological signal; and c) monitoring physical movement of the target body in a detection mode corresponding to the output signal of step b).

14. The method according to claim 13, wherein the physiological signal is any one of heart beat, blood pressure, blood pulse, ECG, EMG, and SPO₂.

15. The method according to claim 13, wherein step b) further comprises the steps of: i) detecting the abnormal occurrence of the physiological signal; and ii) generating a mode-selection signal for use in step c) to determine the detection mode.

16. The method according to claim 13, wherein the detection mode is any one of off, sleep, doze, normal and active modes.

17. The method according to claim 13, wherein step c) further comprises the steps of: i) monitoring an acceleration of the target body; ii) monitoring a tilting level of the target body; and iii) processing the output signal of steps i) and ii) for detecting the abnormal situation.

18. The method according to claim 13, further comprising a step of: d) monitoring a change of environment in which the target body is located; wherein step c) is further configured to monitor the physical movement of the target body in a detection mode corresponding to the output signals of steps b) and d).

19. The method according to claim 13, further comprising a step of: e) transmitting the output signal of step c) in accordance with the output signal of step b).
